(19) United States
(12) Patent Application Publication
(10) Pub. No.: US 2017/0333447 A1
(43) Pub. Date: Nov. 23, 2017

(54) HORMONE DELIVERY SYSTEM AND METHOD

(71) Applicant: Karan Y. Baucom, Overland Park, KS (US)

(72) Inventor: Karan Y. Baucom, Overland Park, KS (US)

(21) Appl. No.: 15/671,799

(22) Filed: Aug. 8, 2017

Related U.S. Application Data
(63) Continuation-in-part of application No. 15/231,448, filed on Aug. 8, 2016.
Application No. 15/231,448 is a continuation-in-part of application No. 14/189,801, filed on Feb. 25, 2014, now abandoned.
Application No. 14/189,801 is a continuation-in-part of application No. 12/818,798, filed on Jun. 18, 2010, now Pat. No. 8,658,628.
(60) Provisional application No. 61/218,301, filed on Jun. 18, 2009.

(51) Int. Cl. A61K 31/57; A61K 31/568; A61K 31/565; A61K 31/519; A61K 31/404; A61K 31/198; A61K 31/197; A61K 31/714; A61K 31/194

(52) U.S. Cl. CPC A61K 31/57; A61K 31/714; A61K 31/568; A61K 31/565; A61K 31/194; A61K 31/404; A61K 31/198; A61K 31/197; A61K 31/519

(57) ABSTRACT

A pre-packaged hormone delivery system and method are provided for administering bioidentical human hormones using a combination of modalities for the treatment of human physiological conditions. A combination of indoles, glutathione, methylating agents, glucuronidating agents, sulfating agents, acetylating agents and glutathione are used in combination with bio-identical progesterones and estrogens to safely remove the estrogen from the system as a standard step in the hormone treatment system process.

### CROSS-REFERENCE TO RELATED APPLICATIONS

This application is a continuation-in-part of and claims priority in U.S. patent application Ser. No. 15/231,448, filed Aug. 8, 2016, which is a continuation-in-part of and claims priority in U.S. patent application Ser. No. 14/189,801, filed Feb. 25, 2014, which claims priority in and is a continuation-in-part of U.S. patent application Ser. No. 12/818,798, filed Jun. 18, 2010, now U.S. Pat. No. 8,658,628, issued Feb. 25, 2014, which claims priority in U.S. Provisional Patent Application No. 61/218,301, filed Jun. 18, 2009, all of which are incorporated herein by reference.

### BACKGROUND OF THE INVENTION

### 1. Field of the Invention

The present disclosed technology relates generally to the administration of human hormones, and in particular to various delivery methods of bioidentical hormone formulations.

### 2. Description of the Related Art

Hormones are chemical substances produced by cells and organs of the body that affect organs and body systems. Hormones are important for cardiovascular maintenance, musculature, the skeletal system, and cognitive functioning. The body's production of hormones and how the body reacts to hormones affect the well-being of individuals and the aforementioned body systems.

The body has three general categories of sex hormones; androgens (ex. testosterone), estrogens (estradiol and estrone), and progestagens (ex. progesterone). Particular female sex hormones and their associated organs are, for example, testosterone (ovaries), estrone and estradiol (ovaries), and progesterone (ovaries and placenta). Testosterone promotes the growth and maintenance of the skeletal system, musculature, and connective tissues, to name a few. Estradiol and estrone principally affect the female reproductive system. Progesterone affects the female menstrual cycle, and maintenance of pregnancy. Hormone deficiencies caused by aging, disease states, exogenous and endogenous environmental conditions, and certain prescribed medications can upset the balance of sex hormones within the body and affect general well-being, lifespan, quality of life, and may lead to disease states as well. Therefore, in order to counter the negative effects of hormone deficiencies, patients are often prescribed hormone replacement therapy (HRT) by their treating physicians.

HRT is a system of treatment using either synthetic sex hormones, or bioidentical sex hormones to treat the effects of diminished sexual hormone levels in peri-menopausal, menopausal, and post-menopausal women. Synthetic sex hormones are the predominant type of hormone proscribed in HRT. The types of techniques used in HRT to deliver sex hormones include pills, capsules, gels, creams, patches and troches. Use of synthetic sex hormones in HRT comes with significant problems such as heart problems, cancers and other undesirable side effects. Furthermore, the type and amount of sex hormone administered is limited by dosing regimens associated with the available delivery techniques. Patient selection of desired delivery techniques and source of sex hormones will increase the effectiveness of therapy and compliance. Moreover, the ability of a physician to tailor the amount of sex hormone delivered with each dosing regime will increase the effectiveness of HRT. Therefore, there is a need for a system of HRT that avoids the undesirable side-effects of synthetic hormone treatments, and that provides the patient with a range of techniques for administering the hormones to maximize well-being and maintenance of body systems.

Heretofore there has not been available a hormone delivery system with the advantages and features of the disclosed technology.

### SUMMARY OF THE INVENTION

A hormone delivery system is disclosed providing for administration of bioidentical human hormones using a combination of modalities for the treatment of human physiological conditions where treatment by hormone therapy is indicated. Bioidentical estrogen, progesterone and androgen preparations may be administered individually, or in combination to a patient using one or more modalities such as transdermal absorption or ingestion. The hormone delivery system can be used to treat pre-menstrual tension syndrome, peri-menopause, menopause, post-menopause, progesterone deficiency, estrogen dominance, libido issues, and birth control.

When using bioidentical hormones for the efficacy of treating peri-menopausal and menopausal women, it is best to simulate the natural menstrual cycle. Utilizing estrogen throughout the 28 day cycle (from day 1 to day 28), and introducing progesterone as early as day 10 or as late as day 14, can produce these results. During tests, this resulted in producing a period, primarily during the last 5 days of the month. The progesterone portion of the cycle dominates the shedding or secretory phase of the process. The intensity would vary depending on the dosage of estrogen based upon its proliferation of the endometrial lining.

A “steady state” occurs where there is little or no bleeding during the secretory phase. This is based upon the endometrial layer being so scant that the progesterone does not induce bleeding. Previously it was a concern that the endometrial layer would remain and become exposed to the estrogenic component. However, as long as progesterone is also being introduced, there is a ratio that results in an equilibrium wherein the endometrial layer neither grows nor sheds.

Estrogen dominance can occur when large doses of estrogen are predominantly utilized without being balanced out with progesterone. Inducement of the cycle with the use of progesterone with the intact uterus can help to prevent endometrial hyperplasia. Therefore, any female patient utilizing estrogen in a delivery system should also balance that with progesterone.

The type of progesterone used is also important. Oral micronized progesterone and various progestogens are available. Progestogens include natural progesterone and synthetic progestins.

Existing solutions provided by physicians do not incorporate both estrogen and progesterone in a physiological manner that can be simply dosed to the patient. A preferred solution would include a distribution system that is pre-packaged and self-explanatory to the patient.

An embodiment of the present invention presents a hormone delivery system (HDS) which provides the hormones to the patient in a biologically, physiological manner which simulates the menstrual cycle. These hormones can be presented in a number of ways. Transdermal applications include patches, gels, creams, or some combination thereof. The estrogen must be presented in a transdermal application, but the progesterone could also be presented orally, such as in a capsule.

If issues arise, such as breast tenderness and extensive bleeding, this can alert the patient and the treating physician to mechanical causes (e.g. fibroids, endometriosis, or andenomyosis) or that the estrogenic component is being given in such a way that, combined with the patient's own endogenous estrogens, have created estrogen dominance. The remedy is to decrease the estrogenic component of the HDS.

The advantage of the present invention is to provide an HDS that is modifiable by the treating physician by adjusting the dosages. As an example, using gels and creams, the physician can prescribe 25 milligrams per application. The hormones being provided through the HDS are well suited for these variations.

### BRIEF DESCRIPTION OF THE DRAWINGS

The drawings constitute a part of this specification and include exemplary embodiments of the present invention illustrating various objects and features thereof.

FIG. 1 box diagram illustrating the relationship between various elements of an embodiment of the present invention.

FIG. 2 is a chart diagramming a timeline based upon various elements and aspects of the present invention.

FIG. 3 is a diagrammatic representation of a sample Steriodogenic pathway.

FIG. 4 is a pie chart diagramming an optimal balance of estrogen metabolites.

FIG. 5 is a pie chart diagramming an optimal balance of estrogen metabolites.

### DETAILED DESCRIPTION OF THE PREFERRED EMBODIMENTS

### I. Introduction and Environment

As required, detailed aspects of the disclosed technology are disclosed herein; however, it is to be understood that the disclosed aspects are merely exemplary of the disclosed subject matter, which may be embodied in various forms. Therefore, specific structural and functional details disclosed herein are not to be interpreted as limiting, but merely as a basis for the claims and as a representative basis for teaching one skilled in the art how to variously employ the disclosed technology in virtually any appropriately detailed structure.

### II. Embodiment or Aspect of the Hormone Delivery System

The hormone delivery system embodying the principles of the disclosed technology provides for administration of compositions containing bioidentical human hormones in an amount sufficient to provide therapeutic effect, using a combination of modalities, for the treatment of human physiologic conditions. Examples of specific bioidentical hormones which may be used include, but are not limited to estrogens (estrone, estradiol), progesterone, and androgens (testosterone, androstenedione, dihydrotestosterone (DHT), dehydroepiandrosterone (DHEA)) isomers and derivatives thereof, and combinations thereof. Examples of specific modalities which may be used include, but are not limited to topical preparations (gel, cream), ingested articles (tablet, lozenge, capsule, troches), and articles for transdermal absorption of hormone preparations (transdermal patch, impregnated matrices). The type and amount of hormones involved in the various bioidentical human hormone compositions, and the modalities used varies depending independently, or in conjunction with, the physiologic sequence based on the normal menstrual cycle pattern, and specific clinical syndromes involved. A single bioidentical hormone or a combination of bioidentical hormones may be used with any particular modality.

The hormone delivery system may be used for the treatment of conditions related to hormone imbalances or deficiencies where treatment by hormone therapy is indicated. For example, the types of conditions for which the hormone delivery system may be used include, but are not limited to: pre-menstrual tension syndrome; peri-menopause, menopause, post-menopause; progesterone deficiency; estrogen dominance; and libido issues. Additional treatment measures include providing birth control. In conjunction with treatments using estrogens, the hormone delivery system is used in conjunction with indole-3-carbinol, di-indole methane, or flax seed to protect biochemically from hydroxylation of estrogen.

### III. Alternative Embodiment or Aspect of the Hormone Delivery System 2

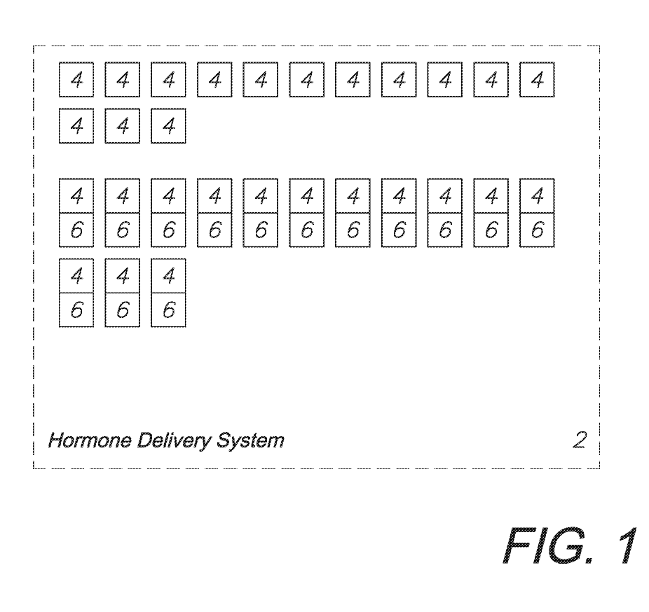

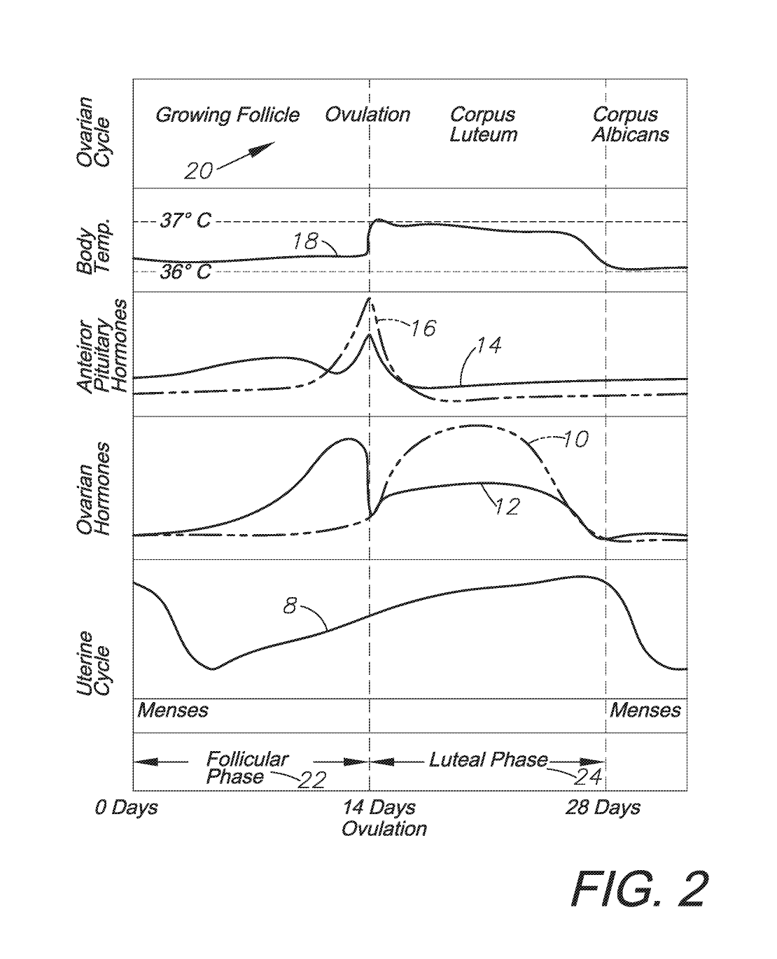

As shown in FIG. 1, a pre-packaged hormone delivery system (HDS) 2 comprising another embodiment or aspect of the disclosed technology can be used for treating peri-menopause, menopause and post-menopause. The bioidentical human hormones estrogens 4 (estrone, estradiol) and progesterone 6 using a combination of modalities mentioned above are administered to a patient in a sequential format following the human physiological twenty-eight day menstrual cycle as diagrammed in FIG. 2. Any combination of patches, capsules and other types of delivery systems can be utilized, provided the menstrual cycle is simulated.

Various combinations of estrogen/progesterone can be administered as a cream with suitable proportions and dosage ranges.

The present invention is pre-packaged for clinical efficiency. This ensures the proper dosage without requiring the physician to mix and match the estrogen and the progesterone doses separately. The hormones being applied through the delivery system are FDA approved in a therapeutic fashion for hormone replacement therapy. Examples include 17-B estradiol, progesterone, progestogens, and progestins.

The delivery system is intended for involving estrogen throughout the standard menstrual cycle (from day one through the entire month) and utilizing progesterone as early as day ten or as late as day fourteen. This ensures progesterone is initiated in addition to the estrogen for the remainder of the cycle. This results in a period primarily during the final 5 days of the month based upon the dosage of estrogen, as it is the estrogen component that proliferates the endometrial lining.

The shedding of secretory phase, as shown in FIG. 2, is under the dominance of the progesterone portion of the cycle, initiating the menstrual cycle. The bleeding can be controlled based upon how much estrogen is provided and as to how the endometrial lining responded in its proliferation.

The present invention simulates the physiological menstrual cycle as its template, mimicking the normal rhythmic delivery of the sex steroids. FIG. 2 illustrates this in some detail. The graph element 8 illustrates the uterine cycle; namely, the thickness of the endometrial lining during a typical cycle. Element 10 graphs the progesterone hormone level over that cycle, and element 12 illustrates the estradiol level. Similarly, element 14 illustrates the follicle-stimulating hormone (FSH) while element 16 represents the lutenizing hormone (LH). Body temperature along the cycle is illustrated at element 18. The phases of the cycle are listed at 20. The follicular phase is represented at 22 and the luteal phase at 24.

Summarizing, the hormone delivery system of this embodiment provides estrogen and progesterone replacements. The estrogen would be provided via a topical solution such as a gel or cream, but the progesterone could be provided either through a micronized oral progesterone (e.g., capsule, sub lingual, troche, or pill) or through a topical, preferably vaginal, gel, cream, or transdermal spray. Dosage ranges for the progesterone can range between 20 milligrams through 300 milligrams.

The 28 day cycle may initiate the progesterone component as early as day 10 or as late as day 14. The component is used cyclically in association with continuous bioidentical estrogen.

By way of example and without limitation, estrogen can be administered as a patch, gel or cream in suitable dosages of, for example, 0.25 mg, 0.375 mg, 0.5 mg, 0.75 mg and 1.0 mg. Progesterone can be administered as a patch, gel, cream, capsule or sublingual in suitable dosages of, for example, 25 mg, 50 mg, 75 mg, 100 mg, 200 mg and 300 mg. Di-indole methane/3-indole carbinol (Dim/3IC) can be administered as 400 mg tablets or gels. However, estrogen-dominant patients should only be given progesterone during days 1-25 of their menstrual cycles. If any estrogen is used, an option is to utilize 3IC/Dim for pathway protection for increasing 2-meo/2 hydroxylation excretion. It is generally important for the patient to excrete estrogen.

Support for the concepts disclosed herein can be found in the following list of references, where are incorporated herein by reference:
Cameron, Sharon T. et al., Continuous Transdermal Oestrogen and Interrupted Progestogen as a Novel Bleed-Free Regimen of Hormone Replacement Therapy for Postmenopausal Women, British Journal of Obstetrics and Gynaecology, October 1997, Vol. 104 pp. 1184-1190.
Campagnoli, C. et al., Progestins and Progesterone in hormone Replacement Therapy and the Risk of Breast Cancer, Journal of Steroid Biochemistry and Molecular Biology, July 2005 Vol. 96 No. 2 pp. 95-108.
Role of Progestogen in Hormone Therapy for Postmenopausal Women: Position Statement of the North American Menopause Society (Retired), Menopause: The Journal of the North American Menopause Society, Vol. 10 No. 2, pp. 113-132.
William Faloon, Surprise Findings in Estrogen Debate, Life Extension Magazine, November 2013.
   IV. Alternative Embodiment Hormone Delivery System for Treatment of Libido Issues with Menopause

An alternative embodiment of the present invention seeks to treat libido issues, specifically but not exclusively during menopause, by providing a combination of estrogen, testosterone, and progesterone to the patient. Testosterone will be prescribed to the patient from day 7 through day 20 of the 28 day cycle.

### V. Alternative Embodiment Hormone Delivery System for Treatment of PMDD, PMS, and Estrogen Dominance

An alternative embodiment of the present invention uses a combination of estrogen and progesterone at dosages of 25 mg, 50 mg, 100 mg, or 200 mg to treat PMDD, PMS, and/or estrogen dominance. This combination can also be used to treat insomnia in menopausal and post-menopausal women.

VI. Alternative Embodiment Hormone Delivery System for Treatment of Vaginal Dryness, Atrophy, and/or Dyspareunia

An alternative embodiment of the present invention uses a combination of estradiol and testosterone to treat vaginal dryness, atrophy, and/or dyspareunia. A combination of 2 mg/ml of estradiol and 5 mg/ml of testosterone is applied intravaginally 2-3 times per week. This is not to be used for menopausal systemic therapy.

VII. Alternative Embodiment Hormone Delivery System for Treatment of Severe Menstrual Pain (e.g. Dysmenorrhea and Premenstrual Dysmorphic Syndrome (“PMDS”))

An alternative embodiment of the present invention uses a bio-identical progesterone provided to the patient for a twenty-five (25) day cycle during a month. Bio-identical estrogen may also be sequentially added during this process. These applications of bio-identical hormones can be used to treat Dysmenorrhea and PMDS. Dysmenorrhea is the medical term for the painful cramps that may occur immediately before or during the menstrual cycle.

There are two types of Dysmenorrhea: primary and secondary. Primary dysmenorrhea is another name for common menstrual cramps. The pain or cramping is felt in the lower abdomen of back. The pain or cramping starts shortly before or at the onset of the period and may continue for one to three days. It can be associated with premenstrual dysmenorrhea syndrome, where a more severe form of dysmenorrhea can occur. Symptoms associated with this form can be depression, anxiety, mood disorders, irritability occurring at least two weeks prior to menses.

Secondary dysmenorrhea is pain caused by a disorder in the woman reproductive organs. These cramps begin earlier with the menstrual cycle and last longer. Symptoms of each include: aching pain in the abdomen (sometimes severe); pressure in the abdomen; pain in the hips, lower back, and inner thighs due to cramping; upset stomach; and loose stools.

Progesterone for this purpose may be transmitted transdermally or orally. The oral distribution of the progesterone could be combined with estrogen in a sequential format. In a preferred embodiment, a delivery system would include a package with 25 pills or some other oral administration of the hormones. These would be taken by the patient in sequential order, and estrogen may be added to the same pills such that estrogen is introduced sequentially as well.

Bio-identical Progesterone may be provided in a pre-packaged delivery system sequentially dated for a thirty-day cycle, where the progesterone is provided alone for the first 25 days of the thirty-day cycle. Alternatively, bio-identical progesterone may be provided for days 1-30 of the 30 day cycle and bio-identical estrogen introduced in a separate modality (e.g. a separate pill or other modality) from days 14-30. Alternatively, bio-identical estrogen may be provided for days 1-30 of the 30 day cycle and bio-identical progesterone introduced as early as day 10 or as late as day 14 through the end of the 30-day cycle. The treatment should fit the needs of the patient. The pre-packaged hormones are labeled and produced in such a way as to help the patient maintain an orderly cycle of taking the correct hormones on the correct day of the cycle.

VIII. Alternative Embodiment Hormone Delivery System Including Estrogen Removal Through Glucuronidation, Sulfation, Acetylation, and/or Methylation or Use of Glutathione

In an alternative embodiment hormone delivery system and previously disclosed, the use of a glucuronidating, sulfating, acetylating, and/or methylating agent could be included in place of or in combination with the indole (e.g. Di-indole methane or 3-indole carbinol). In addition, glutathione or other powerful anti-oxidants could be used as well in combination with any of these elements. Similar to the function of the Indoles as previously disclosed, these agents can be taken throughout a cycle as described by the present invention to provide a means for eliminating estrogen from the body, where it would otherwise store up harmfully.

Methylation could include, but would not be limited to, the introduction of methyl B12, trimethylglycine, and/or methyltetrahydro folate (MTHF) or other suitable methylating agent. Glucuronidation could be controlled by introducing D-glutarate. Sulfation could be controlled using MSM, broccoli, Brussels sprouts, Ibuprofen, or Acetaminophen. Acetyl could be controlled through acetyl-L carnitine. Finally, Glutathione could be enhanced through using agents, such as N-Acetyl cysteine (NAC), which is a precursor to glutathione and glutathione itself, could be introduced orally, transdermally or nasal spray.

Any of these processes can be used to detoxify products as they are downgraded within the body's liver. Introduction of these elements can be used to safely remove the estrogen and other hormones, androgens, and DHEA which are introduced into the body through the present hormone delivery system in a safe way. The results are similar to the use of 3-indole carbinol (3IC) or Di-indomethane as previously discussed.

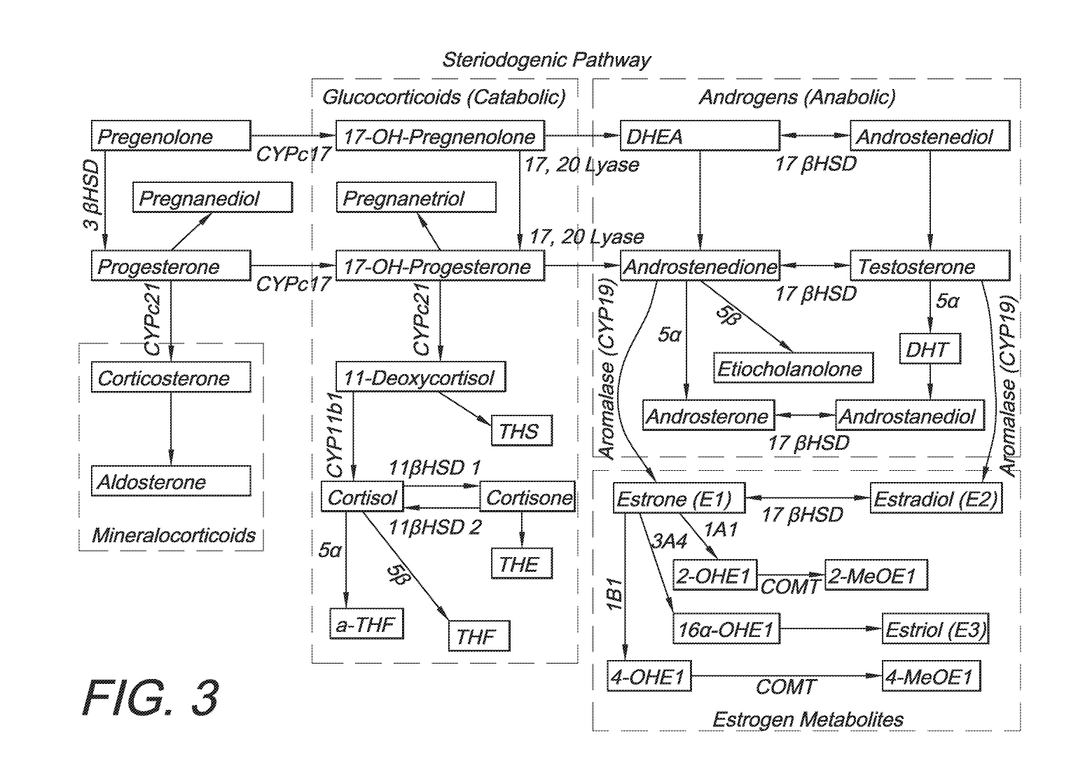

The detoxification pathways that safely allow estrogen, progesterone, testosterone and/or DHEA are as shown in the Steriodogenic Pathway as shown in FIG. 3. It is clear that utilizing the processes such as methylation, glucuronidation, sulfation, glutathione, and acetylation support safe liver detoxification. Therefore the introduction of methyl B12 and methyltetrahydro folate to the hormone delivery system in the form of a sublingual tablet, gel cream, troche, or patch would be inherently beneficial to the patient. Similarly, the use of D-glutarate for glucoronidation in any format of synthetic or natural form in a sub lingual, tablet, capsule, gel cream or patch could be utilized.

Referring to FIG. 3 in more detail, the following legend should be used to identify the Enzymatic steps:

| | |
|---|---|
| 3βHSD | 3beta-Hydroxysteroid dehydrogenase |
| 5α | 5alpha-Reductase |
| 5β | 5beta-Reductase |
| CYP11b1 | 11beta-Hydroxylase |
| 11βHSD | 11beta-Hydroxysteroid dehydrogenase |
| 17βHSD | 17beta-Hydroxysteroid dehydrogenase |
| 17,20 Lyase | 17,20 Desmolase |
| CYPc17 | 17 alpha-Hydroxylase |
| CYP19 | Aromatase |
| CYP21 | 21-Hydroxylase |

Similarly, the following legend should be used to identify the estrogen metabolism steps:

| | |
|---|---|
| 1A1 | Cytochrome p450 1A1 (CYP1A1) |
| 3A4 | Cytochrome p450 3A4 (CYP3A4) |
| 1B1 | Cytochrome p450 1B1 (CYP1B1) |
| COMT | Catechol-O-Methyltransferase |

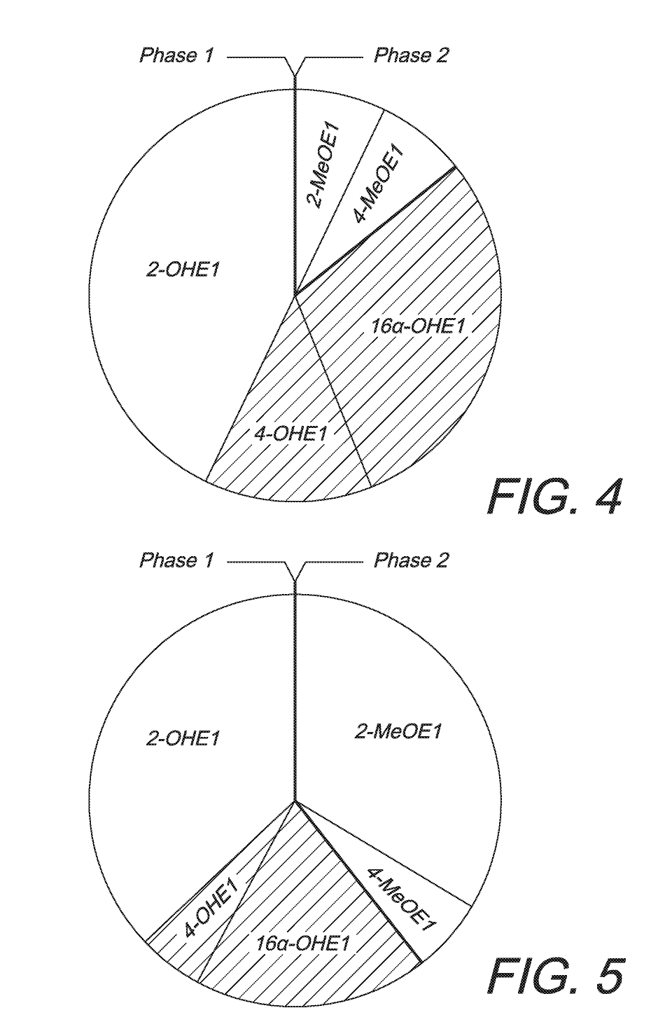

In estrogen metabolism, the 2-Hydroxyestrone/16α-Hydroxyestrone Ratio (16α-OHE1:2-OHE1) can affect the associated risk of breast cancer or prostate cancer. Methylation particularly can affect this outcome in a positive way to reduce the likelihood of cancer. An optimal balance of estrogen metabolites. FIGS. 4 and 5 show charts which represent this optimal balance. The metabolites in the non-hatched portion are considered protective, whereas metabolites in the hatched portion are associated with an increased risk of auto-immune disease, breast, and prostate cancer. In FIGS. 4 and 5, the heavy lines separate the Phase 1 and Phase 2 detoxification pathways.

Phase 2 metabolism is a process whereby a compound is first subject to oxidation reduction or hydrolysis (Phase 1 reactions), which may be associated with bioactivation, and then the functional group created is conjugated to a less toxic or inactive compound. Glucuronides, methylides, sulfides, and the other compounds discussed herein produce this result.

It will be appreciated that the components of the hormone delivery system can be used for various other applications. Moreover, the hormone delivery system can be fabricated in various sizes and dosages and from a wide range of suitable materials, using various manufacturing and fabrication techniques

It is to be understood that while certain aspects of the disclosed technology have been shown and described, the disclosed technology is not limited thereto and encompasses various other embodiments and aspects.

Having thus described the invention, what is claimed as new and desired to be secured by Letters Patent is:

1. A pre-packaged hormone delivery system comprising:
a pre-packaged, predetermined quantity of bio-identical progesterone, wherein said bio-identical progesterone is provided in a modality selected from the list comprising: a transdermal application; and an oral modality;
a pre-packaged, predetermined quantity of an agent provided sequentially in a modality selected from the list comprising: a transdermal application; and an oral modality;
wherein said agent is selected form the list comprising: a glucuronidation agent; a sulfation agent; an acetylation agent; a methylation agent; and glutathione;
said bio-identical progesterone delivered to a patient in predetermined doses during a predetermined cycle; and
said bio-identical progesterone configured to treat a symptom selected from the list comprising: insomnia, menstrual disorder; phases of the menopausal syndrome; pre-menstrual syndrome, perimenopausal symptoms; menopause symptoms; and post menopause symptoms.

2. The pre-packaged hormone delivery system of claim 1, wherein said predetermined cycle is a 25-day cycle, and wherein said menstrual disorder is selected from the list comprising: premenstrual syndrome; and menstrual cramp dysmenorrhea.

3. The pre-packaged hormone delivery system of claim 1, further comprising:
a pre-packaged, predetermined quantity of bio-identical estrogen, wherein said bio-identical estrogen is provided in a modality selected from the list comprising: an oral pill; a sublingual pill; a patch; or a gel;
wherein said bio-identical estrogen is delivered sequentially with said bio-identical progesterone;
wherein said estrogen and said progesterone are contained in separate modalities;
wherein a first 14 daily doses comprise said bio-identical estrogen only; and
wherein a second 14 daily doses comprise both said bio-identical estrogen and said bio-identical progesterone each packaged separately.

4. The pre-packaged hormone delivery system of claim 3, further comprising:
wherein said estrogen is provided in a thirty-day cycle and is packaged accordingly within said pre-packaged delivery system; and
wherein said progesterone is packaged such that it is introduced at a date selected from the list comprising: day 10 of said thirty-day cycle; and day 14 of the thirty-day cycle.

5. The pre-packaged hormone delivery system of claim 3, further comprising:
wherein said progesterone is provided in a thirty-day cycle and is packaged accordingly within said pre-packaged delivery system;
wherein said estrogen is packaged for use during days 1 through 30 of said thirty-day cycle; and
wherein said progesterone is packaged such that it is introduced at a date selected from the list comprising: day 10 of said thirty-day cycle; and day 14 of the thirty-day cycle.

6. The pre-packaged hormone delivery system of claim 3, wherein said bio-identical progesterone is plant based.

7. The pre-packaged hormone delivery system of claim 3, wherein said bio-identical estrogen is derived from plants.

8. The pre-packaged hormone delivery system of claim 3, wherein said bio-identical estrogen is delivered in a dosage amount selected from the list comprising: 0.025 mg; 0.0375 mg; 0.05 mg; 0.075 mg; 0.25 mg; 0.375 mg; 0.5 mg; 0.75 mg; and 1.0 mg.

9. The pre-packaged hormone delivery system of claim 1, further comprising:
wherein said bio-identical progesterone is delivered in a modality selected from the list comprising: oral; transdermal; and vaginally; and
wherein said bio-identical progesterone is provided in an amount selected from the list comprising: 20 mg; 30 mg; 50 mg; 100 mg; 200 mg; and 300 mg; and administered for insomnia, menstrual disorders.

10. The pre-packaged hormone delivery system of claim 3, wherein said bio-identical estrogen is delivered in a dosage amount selected from the list comprising: 025 mg; 0.0375 mg; 0.05 mg; 0.075 mg; and 0.1 mg.

11. The pre-packaged hormone delivery system of claim 3, further comprising:
a pre-packaged, predetermined quantity of bio-identical testosterone, wherein said bio-identical testosterone is provided in a modality selected from the list comprising: transdermally; intramuscularly; and transvaginally;
wherein said bio-identical testosterone is delivered sequentially with said bio-identical progesterone and said bio-identical estrogen;
wherein said bio-identical testosterone is provided from day 7 through day 20 of a 28-day cycle; and
wherein said bioidentical testosterone dosage is provided in a modality and dosage selected from a list comprising:
intramuscularly in a dosage selected from a list comprising: 2.5 mg; 5 mg; 10 mg; 25 mg 100 mg; 150 mg; 200 mg; and 300 mg;
transdermally in a dosage selected from a list comprising: 2.5 mg; 5 mg; 10 mg; 20 mg; 30 mg; 50 mg; 75 mg; and 100 mg; and
intravaginally in a dosage selected from a list comprising: 2 mg; 5 mg; and 10 mg.

12. The pre-packaged hormone delivery system of claim 1, wherein said agent is a glucuronidation agent comprising D-glutarate.

13. The pre-packaged hormone delivery system of claim 1, wherein said agent is a sulfation agent selected from a list comprising: MSM; broccoli; Brussels sprouts; Ibuprofen; and Acetaminophen.

14. The pre-packaged hormone delivery system of claim 1, wherein said agent is methyl B12 in a dosage between 1000 mcg to 10,000 mcg, and wherein said agent is delivered in a frequency selected from a list comprising: daily; and weekly.

15. The pre-packaged hormone delivery system of claim 1, wherein said agent is trimethylglycine in a dosage between 200 mg to 400 mg delivered daily.

16. The pre-packaged hormone delivery system of claim 1, wherein said agent is methyltetrahydro folate (MTHF) in a dose of 1000 mcg delivered daily.

17. The pre-packaged hormone delivery system of claim 1, wherein said agent is an acetylation agent comprising acetyl-L carnitine.

18. The pre-packaged hormone delivery system of claim 1, wherein said agent is delivered in a daily dosage between 500 mg to 1000 mg, and is selected from a list comprising: glutathione N-Acetyl cysteine (NAC).

19. The system of claim 1, further comprising:
a pre-packaged, predetermined quantity of indole provided sequentially in a modality selected from the list comprising: a transdermal application; and or oral modality;
wherein said indole is selected from the list of indoles comprising: Di-indole methane/methane and 3-indole carbinol;
wherein said indole is applied in combination with the progesterone only during days 10-31 of each cycle; and
wherein the daily dosages of said indole are selected from a list of dosages comprising: 100 mg; 200 mg; and 400 mg.

20. The system of claim 1, further comprising:
a pre-packaged, predetermined quantity of indole provided sequentially in a modality selected from the list comprising: a transdermal application; and an oral modality;
wherein said indole is selected from the list of indoles comprising: Di-indole methane and 3-indole carbinol; and
wherein said indole is applied daily throughout the entire cycle and wherein said daily dosages of said indole are selected from a list of dosages comprising: 100 mg; 200 mg; and 400 mg.
